(19) United States
(12) Patent Application Publication
(10) Pub. No.: US 2011/0097748 A1
(43) Pub. Date: Apr. 28, 2011

(54) USE OF CELL LINES TO DETERMINE LEVELS OF EFFICACY OF PHARMACEUTICAL FORMULATIONS

(75) Inventor: Vanja Margareta King, Memphis, TN (US)

(73) Assignee: Warsaw Orthopedic, Inc., Warsaw, IN (US)

(21) Appl. No.: 12/607,199

(22) Filed: Oct. 28, 2009

(51) Int. Cl. G01N 33/53; C12Q 1/02

(52) U.S. Cl. 435/7.92; 435/29

(57) ABSTRACT

The present invention provides methods and assays for determining the efficacy of pharmaceutical compositions. Through the use of cell lines to measure efficacy of a formulation, one can better measure characteristics such as half life, release rate and release profile of a product that may be informative to the FDA. These methods and assays may, for example, be of use when measuring the effect of anti-inflammatory agents on cytokine production that is being considered for local administration.

### BACKGROUND

The measurement of the efficacy of a pharmaceutical formulation is important for many reasons, including not the least of which is to gain approval of a new drug application by the appropriate governmental agency, e.g., in the Untied States of America, the Food and Drug Administration (“FDA”). However, it is important be clear what efficacy means for a particular application. For example, in different circumstances efficacy may refer to one or more of the amelioration of certain symptoms, a release dose, a release rate or a half-life.

For pharmaceuticals that are administered orally, one measure of efficacy is the rate at which certain components of the formulation or metabolites of that formulation appear in the blood stream or are excreted. Unfortunately, this type of measurement is neither practical nor ideal for implants that are designed to deliver active ingredients locally i.e., at or near a specific site.

Accordingly, there is a need for an efficient method that allows the measurement of the efficacy of locally administered formulations.

### SUMMARY

In some embodiments, a method is provided that permits the measurement of the efficacy of a formulation. In some embodiments, an assay is provided that permits the measurement of the efficacy of a formulation.

According to one embodiment there is a method for determining the efficacy of a polymer formulation, wherein the method comprises selecting a cell line, wherein the cell line produces a known phenotype after being exposed to a known stimulus; exposing one or more cells from the cell line to the polymer formulation; measuring an experimental phenotype after exposing the one or more cells to the polymer formulation; determining the efficacy of the polymer formulation, wherein the efficacy is determined based on whether the experimental phenotype is an absolute amount or concentration or a relative amount or concentration that is below or above a preselected level at one or more intervals of time. Any relative measurements may be made to a phenotype expressed in the presence of the stimuli but not the polymer formulation or to a phenotype expressed in the presence of a different polymer formulation with or without the stimulus.

According to another embodiment, there is an assay for measuring the efficacy of a polymer formulation, wherein the assay comprises cells from a cell line; a polymer formulation, wherein the polymer formulation is capable of releasing an active ingredient over a period of more than about two days; and a phenotype detection element, wherein the phenotype detection element is capable of measuring a phenotype of the cells of the cell line at one or more points of time.

Among the advantages of some of the embodiments are that by using cell lines instead of whole animals, one simplifies the process for producing data that satisfies governmental regulatory bodies. This may reduce costs and time that is expended.

### DETAILED DESCRIPTION

Unless defined otherwise, all technical and scientific terms used herein have the same meaning as is commonly understood by one of ordinary skill in the art to which this invention belongs. For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth are reported as precisely as possible. Any numerical value, however, inherently contains certain errors necessarily resulting from the standard deviation found in its respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

According to one embodiment there is a method for determining the efficacy of a polymer formulation, wherein the method comprises selecting a cell line, wherein the cell line produces a known phenotype after being exposed to a known stimulus; exposing one or more cells from the cell line to the polymer formulation; measuring an experimental phenotype after exposing the one or more cells to the polymer formulation; determining the efficacy of the polymer formulation, wherein the efficacy is determined based on whether the experimental phenotype is below or above a preselected level at one or more intervals of time. The determination may be automated. Thus, the measurement of the experimental phenotype may be by a person or machine and then compared to another observed phenotype or known phenotype that is for example, stored in the memory of a computer. Furthermore, any comparison may in some embodiments be controlled by the central processing unit of a computer and output to a digital memory or in a form that is readable by a human such as on a computer screen or printed onto paper.

As used herein, the term “efficacy” refers to the measurement of the desired outcome at a desired time. Thus, efficacy may, for example, be the amount or concentration of a substance that is excreted by the cells at a given time or over a period of time or at multiple times. For example, it may be the measurement of cytokines at one or more of 15 minutes, 30 minutes, 1 hour, 1.5 hours, 2 hours, 6 hours, 8 hours, 12 hours, 18 hours, 24 hours, 48 hours, one week, one month, etc.

As used herein, a “phenotype” is any observed trait or characteristic, e.g., production of protein.

As persons of ordinary skill in the art are aware, different cell lines have different abilities to express different proteins when subject to different stimuli. The term “stimuli” refers to external forces that may induce a response. Thus, by way of example, the stimuli may be physical stress, the presence of absence of nutrients, the presence of absence of chemical agents, the presence or absence of one or more biologic agents or molecules, light or the absence of light, heat or cold, agitation, or combinations thereof.

A non-limiting example of a cell line is RAW264.7, which is a macrophage cell line that will produce cytokines after an inflammatory insult. Examples of cytokines include but are not limited to GM-CSF, IL-1α, IL-1β, IL-2, IL-3, IL-4, IL-5, IL-6, IL-7, IL-8, IL-10, IL-12, IFN-α, IFN-β, IFN-γ, MIP-1α, MIP-1β, TGF-β, TNF-α, and TNF-β.

Other cell lines that are known to persons of ordinary skill in the art include but are not limited to RBL-ZH3 THP-1, EA13.5, U-937, P31/FUJ, J774A1, PEER, MOLT 13, WM 14, AK119, P388, P815 or neutrophil cell lines. As a person of ordinary skill in the art is aware, selection of a cell line should at least in part be based on the ability of the cell line to express a phenotype (or protein) in response to a stimulus of interest.

In some embodiments, the formulation includes one or more growth factors including, but not limited to, isolated Bone Morphogenic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta707) polypeptides or combinations thereof. Some examples include BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18, osteoprotegerin, CTGF-1, CTGF-2, CTGF-4, VEGF-A, VEGF-B, VEGF-C, VEGF-D, VEGF-E, TGF-beta-1, TGF-beta-2, TGF-beta-3, GDF-1, GDF-2, GDF-3, GDF-7, GDF-10, GDF-11, and GDF-15, CDMP-1, CDMP-2, LMP-1, LMP-2, or LMP-3, or combinations thereof.

By way of a further non-limiting example, the polymer formulation may comprise a small molecule. A small molecule is a low molecular weight organic compound that is not a polymer. In some embodiments a small molecule may be a molecule that binds with high affinity to a biopolymer such as protein, nucleic acid, or polysaccharide. A small molecule may alter the activity or function of the biopolymer. In some embodiments, the small molecule has a molecular weight of less than about 800 Daltons, less than about 700 Daltons, less than about 500 Daltons, or less than about 400 Daltons, which allows for the possibility of rapid diffusion across cell membranes so that they can reach intracellular sites of action.

Small molecules can have a variety of biological functions, serving as cell signaling molecules, as tools in molecular biology, and as pharmaceuticals in medicine. These compounds can be natural (such as secondary metabolites) or artificial (such as antiviral drugs); they may have a beneficial effect against a disease (such as drugs) or may be detrimental (such as teratogens and carcinogens).

Examples of small molecules include, but are not limited to, anti-inflammatory agents, anti-infective agents (e.g., antiviral, antibacterial, antifungal agents, etc.), tissue and bone growth factors, pain management medication (e.g., analgesics, anesthetics, etc.) antineoplastic agents, antineoplastic agents, selective H-2 antagonists, nutrients, vitamins, minerals, herbal products, or mixtures thereof. Suitable anti-inflammatory agents to treat and reduce inflammation include both steroidal and non-steroidal anti-inflammatories.

Examples of anti-inflammatory agents include but are not limited to alclofenac; alclometasone dipropionate; algestone acetonide; alendronate sodium; alpha amylase; amcinafal; amcinafide; amcinonide; amfenac sodium; amiprilose hydrochloride; anakinra; anirolac; anitrazafen; apazone; balsalazide disodium; beclomethasone dipropionate; bendazac; benoxaprofen; benzydamine hydrochloride; betamethasone; bromelains; broperamole; budesonide; carprofen; cicloprofen; cintazone; cliprofen; clobetasol propionate; clobetasone butyrate; clopirac; cloticasone propionate; cormethasone acetate; cortisone acetate; cortodoxone; deflazacort; desonide; desoximetasone; dexamethasone dipropionate; diclofenac potassium; diclofenac sodium; diflorasone diacetate; diflumidone sodium; diflunisal; difluprednate; diftalone; dimethyl sulfoxide; drocinonide; endrysone; enlimomab; enolicam sodium; epirizole; etodolac; etofenamate; felbinac; fenamole; fenbufen; fenclofenac; fenclorac; fendosal; fenpipalone; fentiazac; flazalone; fluazacort; fludrocortisone; flufenamic acid; flumizole; flunisolide acetate; flunixin; flunixin meglumine; fluocinonide; fluocinolone acetonide; fluocortin butyl; fluorometholone acetate; fluquazone; flurandrenolide; flurbiprofen; fluretofen; fluticasone propionate; furaprofen; furobufen; halcinonide; halobetasol propionate; halopredone acetate; hydrocortisone; ibufenac; ibuprofen; ibuprofen aluminum; ibuprofen piconol; ilonidap; indomethacin; indomethacin sodium; indoprofen; indoxole; intrazole; isoflupredone acetate; isoxepac; isoxicam; ketoprofen; lofemizole hydrochloride; lomoxicam; loteprednol etabonate; meclofenamate sodium; meclofenamic acid; meclorisone dibutyrate; medrysone; mefenamic acid; mesalamine; meseclazone; methylprednisolone suleptanate; momiflumate; nabumetone; naproxen; naproxen sodium; naproxol; nimazone; nilutamide; olsalazine sodium; orgotein; orpanoxin; oxaprozin; oxyphenbutazone; pamidronate disodium; paramethasone; paranyline hydrochloride; pentosan polysulfate sodium; phenbutazone sodium glycerate; pirfenidone; piroxicam; piroxicam cinnamate; piroxicam olamine; pirprofen; prednazate; prednisolone; prifelone; prodolic acid; proquazone; proxazole; proxazole citrate; rimexolone; romazarit; salcolex; salnacedin; salsalate; sanguinarium chloride; seclazone; sermetacin; sudoxicam; sulindac; suprofen; talmetacin; talniflumate; talosalate; tebufelone; tenidap; tenidap sodium; tenoxicam; tesicam; tesimide; tetridamine; tiopinac; tixocortol pivalate; tolmetin; tolmetin sodium; triamcinelone; triclonide; triflumidate; zidometacin; zomepirac sodium or combinations thereof.

Anti-inflammatory agents include steroidal agents or glucocorticosteroids. Phospholipase A2 (“PLA2”) is a lipolytic enzyme that has been implicated as a possible mediator of inflammation. Specifically, PLA2 hydrolyses the 2-acyl position of glycerophospholipids, liberating free-fatty acids, mainly arachidonic acid. Subsequently, it is believed that arachidonic acid is converted into a variety of proinflammatory eicosanoids. Glucocorticosteroids are known to stop or reduce the suggested mechanisms of inflammation that involves the activation of the arachidonic acid cascade, which results in the liberation of a variety of proinflammatory eicosanoids by inducing lipocortin that inhibits PLA2. This provides a significant advantage over non-steroidal anti-inflammatory agents that enter the cascade much later. Suitable glucocorticosteroids include, but are not limited to, alclometasone diproprionate, alendronate sodium, amcinonide, beclomethasone diproprionate, betamethasone, budesonide, clobetasol propionate, cortisone, dexamethasone, diflorasone diacetate, hydrocortisone, fludrocortisone; flunisolide acetate, fluocinolone acetonide, fluocinonide, fluorometholone acetate, flurandrenolide, halcinonide, medrysone; methylprednisone suleptanate, pamidronate, paramethasone, prednisolone, nilutamide, triamcinelone, or combinations thereof.

In some embodiments, the polymer formulation may comprise an anti-inflammatory compound. Examples of anti-inflammatory compounds include but are not limited to salicylates, diflunisal, sulfasalazine, indomethacin, ibuprofen, naproxen, tolmetin, ketorolac, diclofenac, ketoprofen, fenamates (mefenamic acid, meclofenamic acid), enolic acids (piroxicam, meloxicam), nabumetone, celecoxib, etodolac, nimesulide, apazone, gold, sulindac or tepoxalin; antioxidants, such as dithiocarbamate, and other compounds such as sulfasalazine [2-hydroxy-5-[-4-[C2-pyridinylamino)sulfonyl]azo]benzoic acid], steroids, such as fluocinolone, cortisol, cortisone, hydrocortisone, fludrocortisone, prednisone, prednisolone, methylprednisolone, triamcinolone, betamethasone, dexamethasone, beclomethasone, fluticasone or a combination thereof.

In some embodiments, the formulation comprises clonidine. In some embodiments, the formulation comprises one or more chemotherapeutic agents. Furthermore, the formulation may comprise two or more of any of the aforementioned compounds.

In some embodiments, the polymer formulation may, for example, be a slow release composition such that it is designed to release an active ingredient over a period of at least about 2 days, at least about 3 days, at least about 4 days, at least about 5 days, at least about 6 days, at least about 7 days, at least about 8 days, at least about 9 days, at least about 10 days, at least about 15 days, at least about 20 days, at least about 25 days, at least about 30 days, at least about 35 days, at least about 40 days, at least about 45 days, or at least about 50 days.

In some embodiments, the polymer formulation may for example, be a slow release composition such that it is designed to release an active ingredient over a period of no more than about 60 days, no more than about 50 days, no more than about 40 days, no more than about 30 days, no more than about 25 days, no more than about 20 days, no more than about 15 days, no more than about 10 days, no more than about 8 days, no more than about 6 days, or no more than about 5 days.

The release rate may be controlled in part by the concentration of the active ingredient, the concentration of the polymer, the thickness of the formulation and the composition of the polymer. Furthermore, the polymer formulation may be designed to release a bolus dose followed by a slow release.

In various embodiments, the formulation may comprise a bioerodible, a bioabsorbable, and/or a biodegradable biopolymer that may provide immediate release, or sustained release of the small molecule. Examples of suitable sustained release biopolymers include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), poly(orthoester)s (POE), polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof. As persons of ordinary skill are aware, mPEG may be used as a plasticizer for PLGA, but other polymers/excipients may be used to achieve the same effect. In some embodiments, these biopolymers may also be coated on a drug depot to provide the desired release profile. In some embodiments, the coating thickness may be thin, for example, from about 5, 10, 15, 20, 25, 30, 35, 40, 45 or 50 microns to thicker coatings 60, 65, 70, 75, 80, 85, 90, 95, 100 microns to delay release of the drug from the depot. In some embodiments, the range of the coating on the drug depot ranges from about 5 microns to about 250 microns or 5 microns to about 200 microns to delay release from the drug depot.

In various embodiments, the formulation comprises poly(lactide-co-glycolide) (PLGA), polylactide (PLA), polyglycolide (PGA), D-lactide, D,L-lactide, L-lactide, D,L-lactide-co-ε-caprolactone, D,L-lactide-co-glycolide-co-ε-caprolactone or a combination thereof.

The formulation may be in the form of a drug depot. Various techniques are available for forming at least a portion of a drug depot from the biocompatible polymer(s), therapeutic agent(s), and optional materials, including solution processing techniques and/or thermoplastic processing techniques. Where solution processing techniques are used, a solvent system is typically selected that contains one or more solvent species. The solvent system is generally a good solvent for at least one component of interest, for example, biocompatible polymer and/or therapeutic agent. The particular solvent species that make up the solvent system can also be selected based on other characteristics, including drying rate and surface tension.

Solution processing techniques include solvent casting techniques, spin coating techniques, web coating techniques, solvent spraying techniques, dipping techniques, techniques involving coating via mechanical suspension, including air suspension (e.g., fluidized coating), ink jet techniques and electrostatic techniques. Where appropriate, techniques such as those listed above can be repeated or combined to build up the depot to obtain the desired release rate and desired thickness.

In various embodiments, a solution containing solvent and biocompatible polymer are combined and placed in a mold of the desired size and shape. In this way, polymeric regions, including barrier layers, lubricious layers, and so forth can be formed. If desired, the solution can further comprise one or more of the following: other therapeutic agent(s) and other optional additives such as biological marker(s), etc. in dissolved or dispersed form. This results in a polymeric matrix region containing these species after solvent removal. In other embodiments, a solution containing solvent with dissolved or dispersed therapeutic agent is applied to a pre-existing polymeric region, which can be formed using a variety of techniques including solution processing and thermoplastic processing techniques, whereupon the therapeutic agent is imbibed into the polymeric region.

Thermoplastic processing techniques for forming the depot or portions thereof include molding techniques (for example, injection molding, rotational molding, and so forth), extrusion techniques (for example, extrusion, co-extrusion, multi-layer extrusion, and so forth) and casting.

Thermoplastic processing in accordance with various embodiments comprises mixing or compounding, in one or more stages, the biocompatible polymer(s) and one or more of the following: the active ingredients (e.g., clonidine), optional additional therapeutic agent(s), radiographic agent(s), and so forth. The resulting mixture is then shaped into an implantable drug depot. The mixing and shaping operations may be performed using any of the conventional devices known in the art for such purposes.

During thermoplastic processing, there exists the potential for the therapeutic agent(s) to degrade, for example, due to elevated temperatures and/or mechanical shear that are associated with such processing. For example, certain therapeutic agents may undergo substantial degradation under ordinary thermoplastic processing conditions. Hence, processing is preferably performed under modified conditions, which prevent the substantial degradation of the therapeutic agent(s). Although it is understood that some degradation may be unavoidable during thermoplastic processing, degradation is generally limited to 10% or less. Among the processing conditions that may be controlled during processing to avoid substantial degradation of the therapeutic agent(s) are temperature, applied shear rate, applied shear stress, residence time of the mixture containing the therapeutic agent, and the technique by which the polymeric material and the therapeutic agent(s) are mixed.

Mixing or compounding biocompatible polymer with therapeutic agent(s) and any additional additives to form a substantially homogenous mixture thereof may be performed with any device known in the art and conventionally used for mixing polymeric materials with additives.

Where thermoplastic materials are employed, a polymer melt may be formed by heating the biocompatible polymer, which can be mixed with various additives (e.g., therapeutic agent(s), inactive ingredients, etc.) to form a mixture. A common way of doing so is to apply mechanical shear to a mixture of the biocompatible polymer(s) and additive(s). Devices in which the biocompatible polymer(s) and additive(s) may be mixed in this fashion include devices such as single screw extruders, twin screw extruders, banbury mixers, high-speed mixers, ross kettles, and so forth.

Any of the biocompatible polymer(s) and various additives may be premixed prior to a final thermoplastic mixing and shaping process, if desired (e.g., to prevent substantial degradation of the therapeutic agent among other reasons).

For example, in various embodiments, a biocompatible polymer is precompounded with a biological marker (e.g., dye, label, tag, etc.) under conditions of temperature and mechanical shear that would result in substantial degradation of the therapeutic agent, if it were present. This precompounded material is then mixed with therapeutic agent (e.g., clonidine) under conditions of lower temperature and mechanical shear, and the resulting mixture is shaped into the active ingredient containing drug depot. Conversely, in another embodiment, the biocompatible polymer can be precompounded with the therapeutic agent under conditions of reduced temperature and mechanical shear. This precompounded material is then mixed with, for example, a biological marker, also under conditions of reduced temperature and mechanical shear, and the resulting mixture is shaped into the drug depot.

The conditions used to achieve a mixture of the biocompatible polymer and therapeutic agent and other additives will depend on a number of factors including, for example, the specific biocompatible polymer(s) and additive(s) used, as well as the type of mixing device used.

As an example, different biocompatible polymers will typically soften to facilitate mixing at different temperatures. For instance, where a depot is formed comprising PLGA or PLA polymer, a radio-opacifying agent (e.g., bismuth subcarbonate), and a therapeutic agent prone to degradation by heat and/or mechanical shear (e.g., clonidine), in various embodiments, the PGLA or PLA can be premixed with the radio-opacifying agent at temperatures of about, for example, 150° C. to 170° C. The therapeutic agent is then combined with the premixed composition and subjected to further thermoplastic processing at conditions of temperature and mechanical shear that are substantially lower than is typical for PGLA or PLA compositions. For example, where extruders are used, barrel temperature, volumetric output are typically controlled to limit the shear and therefore to prevent substantial degradation of the therapeutic agent(s). For instance, the therapeutic agent and premixed composition can be mixed/compounded using a twin screw extruder at substantially lower temperatures (e.g., 100-105° C.), and using substantially reduced volumetric output (e.g., less than 30% of full capacity, which generally corresponds to a volumetric output of less than 200 cc/min). It is noted that this processing temperature is well below the melting points of certain active ingredients, such as an anti-inflammatory and analgesic (e.g., clonidine) because processing at or above these temperatures will result in substantial therapeutic agent degradation. It is further noted that in certain embodiments, the processing temperature will be below the melting point of all bioactive compounds within the composition, including the therapeutic agent. After compounding, the resulting depot is shaped into the desired form, also under conditions of reduced temperature and shear.

In other embodiments, biodegradable polymer(s) and one or more therapeutic agents are premixed using non-thermoplastic techniques. For example, the biocompatible polymer can be dissolved in a solvent system containing one or more solvent species. Any desired agents (for example, a radio-opacifying agent, a therapeutic agent, or both radio-opacifying agent and therapeutic agent) can also be dissolved or dispersed in the solvents system. Solvent is then removed from the resulting solution/dispersion, forming a solid material. The resulting solid material can then be granulated for further thermoplastic processing (for example, extrusion) if desired.

As another example, the therapeutic agent can be dissolved or dispersed in a solvent system, which is then applied to a pre-existing drug depot (the pre-existing drug depot can be formed using a variety of techniques including solution and thermoplastic processing techniques, and it can comprise a variety of additives including a biological marker and/or viscosity enhancing agent), whereupon the therapeutic agent is imbibed on or in the drug depot. As above, the resulting solid material can then be granulated for further processing, if desired.

Typically, an extrusion processes may be used to form the drug depot comprising a biocompatible polymer(s), therapeutic agent(s) and radio-opacifying agent(s). Co-extrusion may also be employed, which is a shaping process that can be used to produce a drug depot comprising the same or different layers or regions (for example, a structure comprising one or more polymeric matrix layers or regions that have permeability to fluids to allow immediate and/or sustained drug release). Multi-region depots can also be formed by other processing and shaping techniques such as co-injection or sequential injection molding technology.

In various embodiments, the depot that may emerge from the thermoplastic processing (e.g., pellet, strip, etc.) is cooled. Examples of cooling processes include air cooling and/or immersion in a cooling bath. In some embodiments, a water bath is used to cool the extruded depot. However, where a water-soluble therapeutic agent such as active ingredients is used, the immersion time should be held to a minimum to avoid unnecessary loss of therapeutic agent into the bath.

In various embodiments, immediate removal of water or moisture by use of ambient or warm air jets after exiting the bath will also prevent re-crystallization of the drug on the depot surface, thus controlling or minimizing a high drug dose “initial burst” or “bolus dose” upon implantation or insertion if this is release profile is not desired.

In various embodiments, the drug depot can be prepared by mixing or spraying the drug with the polymer and then molding the depot to the desired shape or disposing it on or in a medical device. In various embodiments, active ingredients are used and mixed or sprayed with the PLGA or PEG550 polymer, and the resulting depot may be formed by extrusion and dried.

The methods of the present invention are designed to measure efficacy and thus the same methodology may be used to test efficacy of the same active ingredient that is present in different formulations. Two formulations that are tested may differ in concentrations of active ingredient by about 5% to about 10%, by about 10% to about 20%, by about 20% to about 30%, by about 30% to about 40%, by about 40% to about 50%, by about 50% to about 60%, by about 60% to about 70%, by about 70% to about 80%, by about 80% to about 95%, by about 95% to about 99%, by about 99% to about 101%, by about 101% to about 150%, by about 150% to about 200%, by about 200% to about 400%, or by about 400% to about 600%. Thus, in some embodiment, they may have all of the same ingredients, but in different amounts, or they may have the same amount of active ingredients, but different amounts of inactive ingredients and thus potentially different release profiles.

When measuring efficacy, one should be careful to delineate which variable is being measured. For example, one may measure a half-life, an effect at a given time, a profile of effect at two or more intervals, a profile of effects over a period of time, e.g., between about 2 days and about 5 days, about 5 days and about 10 days, about 10 days and about 20 days, about 20 days and about 30 days, about 30 days and about 45 days, and about 45 days and about 60 days.

Furthermore, whether measuring a phenotype, one may select a measurement threshold above or below which one infers a satisfactory change in phenotype due to the formulation. This measurement threshold may be an absolute concentration, a concentration relative to a control, an absolute measurement in terms of signal or weight or a measurement of signal or weight in relation to a control.

In some embodiments, when plating the cell line, initial densities may, for example, be from about 1×10² to about 1×10⁴ cells per well. In some embodiments, when plating the cell line, initial densities may for example be from about 1×10² to about 1×10³ cells per well. In some embodiments, when plating the cell line, initial densities may for example be from about 1×10³ to about 1×10⁴ cells per well. The number of wells may for example, be 24, 36, 48, 96, etc. When plating a particular cell line, persons of ordinary skill in the art will be aware of the temperature and other environmental conditions that are conducive to growth for the cell line that was selected.

The measuring of the phenotype may be performed by any methodology that is now known or than comes to be known to persons of ordinary skill in the art that may be useful for the measurement of the phenotype of a cell line and that a person of ordinary skill in the art would appreciate would be useful in connection with the present invention. For example, the measuring may be performed through the use of an ELISA test, which is an example of a phenotype detection element. A “phenotype detection element” is any tool or system that may be used to measure a phenotype. Thus, a phenotype detection element may be a tool or system that measures the presence or absence of a protein, carbohydrate, amino acid, nucleotide, nucleic acid sequence, metabolite or any or substance depending on the design and implementation of the phenotype detection element.

An ELISA test, which is an enzyme-linked immunosorbent assay, is a biochemical technique used in, for example, immunology to detect the presence of an antibody or an antigen in a sample. In simple terms, in ELISA an unknown amount of antigen is affixed to a surface, and then a specific antibody is washed over the surface so that it can bind to the antigen. This antibody is linked to an enzyme, and in the final step a substance is added that the enzyme can convert to some detectable signal. Thus, in the case of fluorescence ELISA, when light of the appropriate wavelength is shone upon the sample, any antigen/antibody complexes will fluoresce so that the amount of antigen in the sample can be inferred through the magnitude of the fluorescence.

Other detection systems may be developed that use spectrophotometric or colorimetric technologies, Western blots, Southern blots and tag and capture systems. Detection may be by any other method that is now known or that comes to be known to persons of ordinary skill in the art and that may be useful in connection with the present invention.

The methods of the present invention may also be used to determine whether a plurality of active ingredients, e.g., at least two, at least three, at least four, at least five, at least six, etc. active ingredients have simple additive, counteracting or synergistic effects. For example, the formulation may contain both an anti-inflammatory compound and a COX-2 inhibitor (e.g., rofecoxib or celecoxib), and then be applied as a single formulation, or two separate formulations may be exposed to the same cells. The experimental phenotype that is observed may be indicative of the degree of efficacy.

According to another embodiment, there is an assay for measuring the efficacy of a polymer formation, wherein the assay comprises cells from a cell line; a polymer formulation, wherein the polymer formulation is capable of releasing an active ingredient over a period of more than about 2 days; and a phenotype detection element, wherein the phenotype detection element is capable of measuring a phenotype of the cell line.

In some embodiments, the polymer formulation is capable of releasing the active ingredient for a period of from about 2 days to about 10 days. In some embodiments, the polymer formulation is capable of releasing the active ingredient for a period of from about 5 days to about 15 days. In some embodiments, the polymer formulation is capable of releasing the active ingredient for a period of from about 10 days to about 25 days. In some embodiments, the polymer formulation is capable of releasing the active ingredient for a period of from about 20 days to about 40 days. In some embodiments, the polymer formulation is capable of releasing the active ingredient for a period of from about 30 days to about 60 days.

In some embodiments, the phenotype detection element is capable of detecting the presence of cytokines.

The methods and assays of the present invention may be used to determine the efficacy of for example, formulations that are being considered for local administration. The contemplated administration may, for example, be a location in, at or near one or more of the spine, hand, wrist, knee, hip, elbow, foot, ankle, etc.

Additionally, in some embodiments, the methods and assays may be useful for measuring the efficacy of systemic formulations, i.e., those formulations that are part of products that are not directed to local use. These applications may be particularly advantageous when studying complex biological effects. For those studies, the drug amount and the elution may not be sufficient to determine the biological effect because as the temporal or pattern of elution may itself affect biology. Thus, cell lines may be used according to the methods and assays described above to determine that a manufacturing process did not alter the biological effect.

The methods of the present invention may be repeated in order to generate sufficient data that may be formatted for submission to, for example, the FDA.

Having now generally described the invention, the same may be more readily understood through the following reference to the following examples, which are provided by way of illustration and are not intended to limit the present invention unless specified.

What is claimed is:

1. A method for determining the efficacy of a polymer formulation, wherein said method comprises
selecting a cell line, wherein said cell line produces a known phenotype after being exposed to a known stimulus;
exposing at least one cell from the cell line to said polymer formulation;
measuring an experimental phenotype after said exposing said at least one cell to said polymer formulation;
determining the efficacy of the polymer formulation, wherein the efficacy is determined based on whether the experimental phenotype is below or above a preselected level.

2. A method according claim 1, wherein the cell line is RAW264.7.

3. A method according to claim 1, wherein the polymer formulation comprises a small molecule.

4. A method according to claim 1, wherein the polymer formulation comprises an anti-inflammatory agent.

5. A method according to claim 4, wherein the anti-inflammatory agent comprises at least one compound selected from the group consisting of salicylates, diflunisal, sulfasalazine, indomethacin, ibuprofen, naproxen, tolmetin, ketorolac, diclofenac, ketoprofen, fenamates (mefenamic acid, meclofenamic acid), enolic acids (piroxicam, meloxicam), nabumetone, celecoxib, etodolac, nimesulide, apazone, gold, sulindac or tepoxalin; antioxidants, such as dithiocarbamate, and other compounds such as sulfasalazine [2-hydroxy-5-[-4-[C2-pyridinylamino)sulfonyl]azo]benzoic acid], steroids, such as fluocinolone, cortisol, cortisone, hydrocortisone, fludrocortisone, prednisone, prednisolone, methylprednisolone, triamcinolone, betamethasone, dexamethasone, beclomethasone, fluticasone or a combination thereof.

6. A method according to claim 1, wherein the polymer formulation comprises clonidine.

7. A method according to claim 1, wherein the polymer formulation is part of a slow release implant.

8. A method according to claim 1, wherein said measuring is performed by an ELISA test.

9. A method according to claim 4, wherein said cells are exposed to said anti-inflammatory agent and to a COX-2 inhibitor.

10. An assay for measuring the efficacy of a polymer formation, wherein said assay comprises:
cells from a cell line;
a polymer formulation, wherein said polymer formulation is capable of releasing an active ingredient over a period of more than about 2 days; and
a phenotype detection element, wherein the phenotype detection element is capable of measuring a phenotype of said cell line.

11. An assay of claim 10, wherein the cell line is RAW 264.7.

12. An assay of claim 10, wherein the polymer formulation comprises an anti-inflammatory agent.

13. An assay do claim 12, wherein the anti-inflammatory agent comprises at least one compound selected from the group consisting of salicylates, diflunisal, sulfasalazine, indomethacin, ibuprofen, naproxen, tolmetin, ketorolac, diclofenac, ketoprofen, fenamates (mefenamic acid, meclofenamic acid), enolic acids (piroxicam, meloxicam), nabumetone, celecoxib, etodolac, nimesulide, apazone, gold, sulindac or tepoxalin; antioxidants, such as dithiocarbamate, and other compounds such as sulfasalazine [2-hydroxy-5-[-4-[C2-pyridinylamino)sulfonyl]azo]benzoic acid], steroids, such as fluocinolone, cortisol, cortisone, hydrocortisone, fludrocortisone, prednisone, prednisolone, methylprednisolone, triamcinolone, betamethasone, dexamethasone, beclomethasone, fluticasone or a combination thereof.

14. An assay of claim 10, wherein the polymer formulation is capable of releasing said active ingredient for a period of from about 2 days to about 10 days.

15. An assay of claim 10, wherein the polymer formulation is capable of releasing said active ingredient for a period of from about 5 days to about 15 days.

16. An assay of claim 10, wherein the polymer formulation is capable of releasing said active ingredient for a period of from about 10 days to about 25 days.

17. An assay of claim 10, wherein the polymer formulation is capable of releasing said active ingredient for a period of from about 20 days to about 40 days.

18. An assay of claim 10, wherein the polymer formulation is capable of releasing said active ingredient for a period of from about 30 days to about 60 days.

19. An assay of claim 10, wherein the phenotype detection element is capable of detecting the presence of cytokines.

20. An assay of claim 10, wherein the phenotype detection element is an ELISA test.
